(19) United States
(12) Patent Application Publication
(10) Pub. No.: US 2024/0017066 A1
(43) Pub. Date: Jan. 18, 2024

(54) METHOD FOR TREATING OR ALLEVIATING BACK PAIN

(71) Applicant: Eberhard Karls Universitaet Tuebingen Medizinische Fakultaet, Tuebingen (DE)

(72) Inventor: Matthias MORGALLA, Tuebingen (DE)

(21) Appl. No.: 17/863,629

(22) Filed: Jul. 13, 2022

(51) Int. Cl. A61N 1/36

(52) U.S. Cl. CPC A61N 1/36062; A61N 1/36071; A61N 1/36171; A61N 1/36175; A61B 2018/1405

(57) ABSTRACT

The present invention relates to a method for treating back pain, wherein over a certain first time period, a first electrical stimulation is applied via an electrode. The first electrical stimulation is a first tonic electrical signal having a consistent frequency of about 2 Hz to about 35 Hz. Via the first tonic electrical signal, a contraction of the patient's both superficial and deeper lying back's muscle groups is caused.

### TECHNICAL FIELD

The present disclosure is directed generally to muscle stimulation by use of spinal cord stimulation devices for inhibiting pain and associated methods. In particular, the present invention discloses a therapeutic method, referred to as “muscle spinal cord stimulation” (MuscleSCS), for alleviating or inhibiting chronic pain syndromes associated with a patient's back, preferably chronic back pain.

### BACKGROUND

Chronic pain is very difficult to be treated, contrary to acute pain, since a direct medical or physical reason for the pain may either not be identifiable or even not be present. One of the most hampering and most common type of chronic pain is chronic back pain.

The term “chronic” in chronic pain is to be understood here as defining the pain as being not clearly causally trackable. This pain does not appear as a consequence of a clearly identifiable singular incident, but develops slowly, persisting and/or increasing over a longer period, usually more than one month. This type of pain, also called pain-syndrome, has lost its natural warning or danger-signalling function, while continuously impacting the patient's quality of life.

Chronic back pain causes large economic loss, since affected individuals have enhanced numbers of sick days, burden the social and economic system and challenge the health system with respect to financial aspects but also regarding the functionality of the system overall.

Since chronic back pain can merely be cured permanently, therapies are usually symptom alleviating or inhibiting treatments, often comprising long-term medication with analgesics, which may lead to unwanted side-effects or negative interferences with other necessary medications, especially in the elderly. Furthermore, analgesics may lose their effectivity over a longer treatment period or even be addictive to the patient.

Physiotherapy might be an option to avoid long-term intake of analgesics or other medications. However, physiotherapy requires a physiotherapist and repeated appointments, thus is a time-consuming, expensive, inflexible treatment method not to be applied immediately, spontaneously in a painful situation.

Surgical procedures are highly invasive and carry a high risk of complications, while their success in alleviating or inhibiting the pain is not guaranteed.

Several neuromodulative methods exist for treatment of chronic back pain e.g., percutaneous electrical nerve stimulation (PENS) or transcutaneous electrical nerve stimulation (TENS). For PENS, acupuncture-like needle probes are placed into the tissue of the patient to deliver electrical signals. TENS is non-invasive, thus electrical signals are applied superficially by the patients according to their preferences. However, TENS can cause skin-irritations and discomfort, which limits the applicable stimulation intensity and duration and thus limits the efficiency of the treatment. Overall, surface-applied electrical muscle stimulators (EMS) as for example neuromuscular stimulators (NMS) or high voltage galvanic stimulators (HVGS) produce only non-specific superficial muscle pumping motion.

Also, nowadays, spinal cord stimulation (SCS) is used to treat chronic back pain. For this purpose, a spinal cord stimulator (SCS) device is surgically placed under the skin, which delivers electrical pulses to the spinal cord. When turned on, the SCS stimulates the nerves in the area where the pain is felt. Pain is reduced because the electrical pulses modify and mask the pain signal from reaching the brain.

Generally, for application of SCS, the patient has implanted epidurally on the spinal cord an electrode/electrodes for delivery of electrical signals to the spinal cord, via which electrical signals from peripheral nerves enter the central nervous system. Usually, additionally to the electrode itself, a pulse generator, connected to the electrode, is also implanted, which controls the parameters of the electrical signals delivered to the patient's spinal cord.

Thus, currently, a magnitude of different types of pain can be treated by drugs, physiotherapy, surgery or broadly by pain therapy. Especially chronic pain is difficult to be treated. In case, traditional therapies do not yield satisfying benefits, neuromodulation can be applied to alter the pain signalling to the central nervous system. For example, in spinal cord stimulation (SCS) an implanted electrode releases electrical signals to the spinal cord for the treatment of pain sensed at various body regions. However, most presently applied SCS methods are indicated to treat neuralgia (neuropathic pain) but not indicated to treat myalgia, e.g. chronic back pain that is caused by muscular tension. Due to the limited treatment options and the heavy impact of chronic back pain onto the patient's quality of life, there is a demand for new methods for treatment of chronic back pain. The present disclosure teaches a new SCS method to treat chronic back pain by electrical stimulation of superficial and deeper laying patient's back muscles.

However, there is still a demand for completion of the toolbox for treatment of chronic back pain to enhance its efficiency and to adopt it for the individual needs of the patients. Thus, the present invention teaches a new method for alleviating or inhibiting back pain in a patient, enabling the treatment of myalgia and thus extending the use and efficiency of prior art devices and methods.

### SUMMARY

With the present invention a method for treating or alleviating back pain in a patient is provided, the patient having epidurally implanted an electrode, wherein the method comprises the steps of:—applying, over a certain first time period, a first electrical stimulation via an electrode, wherein said first electrical stimulation is a first tonic electrical signal having a consistent frequency of about 2 Hz to about 35 Hz, preferably from about 2 Hz to about 20 Hz, and, via the first tonic electrical signal, causing a contraction of the patient's both superficial and deeper lying back's muscle groups; —optionally repeating the applying of the first electrical stimulation; and thereby alleviating or eliminating the back pain.

With the method according to the invention, when using SCS electrodes, muscles in the back area can be stimulated by applying specific stimulation parameters, which allows a massaging effect in the back area. In turn, additional muscle tensions in the back area can be reached and the pain connected therewith alleviated. Studies were performed to show and prove this effect of additional pain relief and are presented herein.

The common spinal cord stimulation (SCS), as it is known in pain therapy today, can be improved by this new method: The prior art devices and methods are suitable for treatment of neuralgia. Said neuralgia is the perception of pain caused e.g., by unphysiological compression or stretching of nerves. Thus, said neuralgia is caused by defects affecting the pain-signalling nerves as such. However, other types of pain, especially myalgia, are not efficiently to be treated by the prior art. Said myalgia is caused by defects within the muscle tissue, which may be caused e.g., by the overuse of a certain muscle group. Within the medical context of the present invention i.e., chronic back pain, myalgia may occur as a consequence of muscle tension or long-term malposition of the patient's body e.g., during work.

Thus, with the method of the invention muscles are actively stimulated via motoneurons, which are efferent neurons, while SCS methods of prior art stimulate afferent neurons to modulate pain-sensation.

The disclosed invention is a method for treating chronic pain in patients, preferably chronic back pain. The patient has implanted outside the spinal cord on the dura in the spinal canal an electrode, which may be part of a standard SCS device. Usually, additionally to the electrode itself, a pulse generator, connected to the electrode, is also implanted, which controls the parameters of the electrical signals delivered to the patient's spinal cord. The configuration of said parameters can be described as a stimulation program.

Such stimulation programs describe sequences of electrical signals, said electrical signal consisting of pulses being defined by a certain amplitude and pulse width. Sequences of said pulses are defined by a frequency and by inter-pulse intervals. It can be distinguished between “tonic” stimulation and “burst” stimulation. A “tonic” electrical signal is to be understood as an electrical signal whose parameters do not vary over time, by means the frequency, amplitude and pulse width stay consistent. A “burst” electrical signal is to be understood as an electrical signal whose parameters do vary over time, by means the frequency, amplitude and/or pulse width do not stay consistent, but vary over time in between a certain range of values and said pulses are delivered in groups, which are termed “burst”.

The “burst stimulation” is the delivery of an electrical signal consisting of groups of electrical pulses, called bursts, with a wavelike variation of frequency, amplitude and/or pulse width, disclosed e.g., in US 2021/0260365 A1, which is herewith incorporated by reference.

According to one aspect, in the method of the invention the first tonic electrical signal is applied at a consistent amplitude of about 0.25 mA to about 30 mA.

According to a further aspect, in the method of the invention, the first tonic electrical signal has a consistent frequency of about 2 Hz to about 35 Hz, preferably of about 2 Hz to about 20 Hz, preferably of 2 Hz to about 10 Hz; or preferably of 2 Hz, 3 Hz, 4 Hz, 5 Hz, 6 Hz, 7 Hz, 8 Hz, 9 Hz, or 10 Hz.

Also, according to an aspect, the first tonic electrical signal has a consistent pulse width of about 10 μs to about 500 μs.

According to a further aspect, in the method of the invention, the first tonic electrical signal can be stopped at any time, i.e. after a certain time period, and the stimulation is paused for 1-600 seconds, and then be resumed again, producing periods of ON-stimulation and periods of OFF-stimulation.

The amplitude, frequency and pulse width of the first tonic electrical signal as applied in the method of the invention have been proven to be effective for carrying out the method of the invention and alleviating pain.

According to a further aspect, the method of the invention further comprises a step of—applying, over a certain second time period, a second electrical stimulation via the electrode, wherein said second electrical stimulation is a BurstDR electrical signal, delivering groups of pulses at a frequency of 40 Hz and intraBurstDR 500 Hz and at amplitudes lower than the tonic electrical signal, wherein said groups of pulses are separated by a pulse-free period, wherein said BurstDR electrical signal does not cause muscle back contraction; —optionally repeating the applying of the second electrical stimulation; thereby further improving the alleviating or eliminating the back pain.

BurstDR stimulation has been first described by DeRidder et al. (“Burst spinal cord stimulation for limb and back pain”, World Neurosurg. 2013; 80(5):642-649), and is a waveform made up of closely spaced, high-frequency electrical impulses delivered in packets, which are followed by a quiescent period or interburst interval. Additional disclosures can be found in de Vos et al. (“Burst spinal cord stimulation evaluated in patients with failed back surgery syndrome and painful diabetic neuropathy”, Neuromodulation 2014; 17(2):152-159), D'Soura and Strand, (“Neuromodulation With Burst and Tonic Stimulation Decreases Opioid Consumption: A Post Hoc Analysis of the Success Using Neuromodulation With BURST (SUNBURST) Randomized Controlled Trial”, Neuromodulation 2021; 42(1):135-141; and Deer et al., (“Success Using Neuromodulation With BURST (SUNBURST) Study: Results From a Prospective, Randomized Controlled Trial Using a Novel Burst Waveform”, Neuromodulation, 2018; 21(1):56-66), the contents of which are herein incorporated by reference.

In an aspect of the method of the invention, the second electrical stimulation is applied before or after a first electrical stimulation.

In another aspect of the method of the invention, the second electrical stimulation is repeatedly applied before or after repeatedly applying the first electrical stimulation.

Also, in a further aspect, of the method of the invention, the second electrical stimulation is applied in alternating repeats with the first electrical stimulation.

Further, according to an aspect of the method of the invention, the BurstDR electrical signal delivers groups of pulses at a frequency of 40 Hz and intraBurstDR of 500 Hz and at amplitudes between about 0.05 mA and 1 mA, and a duration of 1000 μs per impulse.

In another aspect, the method of the invention further comprises a step of—applying, over a certain second time period, a second electrical stimulation via the electrode, wherein said second electrical stimulation is a second tonic or high frequency electrical signal having a consistent frequency between about 40 Hz and 10000 Hz, wherein said second tonic electrical signal does not cause muscle back contraction; —optionally repeating the applying of the second electrical stimulation; thereby further improving the alleviating or eliminating the back pain.

According to this aspect, the first electrical stimulation does cause muscle back contraction, wherein the second electrical stimulation does not cause muscle back contraction, but treats the neuralogiform pain.

In an aspect of the method of the invention, the certain first time period is from about 1 minute to about 120 minutes, preferably from about 10 minutes to about 30 minutes.

Also, in another aspect of the method of the invention, the certain second time period is from about 10 minutes to about 22 h.

According to an aspect of the method of the invention, the electrode is implanted at a patient's vertebral level between C1 to T12, preferably between T7 to T9.

In the method of the invention, according to an aspect, the electrodes are selected from a plate electrode and a rod-electrode, preferably a lamitrode.

The invention also concerns a method for treating or alleviating back pain in a patient, the patient having epidurally implanted a plate electrode, the method comprising the steps of:—applying, over a certain first time period, a first electrical stimulation via the plate electrode, wherein said first electrical stimulation is a first tonic electrical signal having a consistent frequency of about 2 Hz to about 35 Hz, and, via the first tonic electrical signal, causing a contraction of the patient's both superficial and deeper lying back's muscle groups; —optionally repeating the applying of the first electrical stimulation; —applying, over a certain second time period, a second electrical stimulation via the plate electrode, wherein said second electrical stimulation is a BurstDR electrical signal, delivering groups of pulses at a frequency between about 40 Hz and intraBurstDR of 500 Hz, and at amplitudes lower than the tonic electrical signal, wherein said groups of pulses are separated by a pulse-free period, wherein said BurstDR electrical signal does not cause muscle back contraction, —optionally repeating the applying of the second electrical stimulation, thereby alleviating or eliminating the back pain.

According to an embodiment of this method, the consistent frequency of the first tonic signal is about 2 Hz to about 20 Hz, preferably of 2 Hz to about 10 Hz; or preferably of 2 Hz, 3 Hz, 4 Hz, 5 Hz, 6 Hz, 7 Hz, 8 Hz, 9 Hz, or 10 Hz.

Further, the invention concerns a method treating or alleviating back pain in a patient, the patient having epidurally implanted a rod-electrode, the method comprising the steps of:—applying, over a certain first time period, a first electrical stimulation via the rod-electrode, wherein said first electrical stimulation is a first tonic electrical signal having a consistent frequency of about 2 Hz to about 35 Hz, and, via the first tonic electrical signal, causing a contraction of the patient's both superficial and deeper lying back's muscle groups; —optionally repeating the applying of the first electrical stimulation; —applying, over a certain second time period, a second electrical stimulation via the rod-electrode, wherein said second electrical stimulation is a BurstDR electrical signal, delivering groups of pulses at a frequency of between about 40 Hz and intraBurstDR of 500 Hz and at amplitudes lower than the tonic electrical signal, wherein said groups of pulses are separated by a pulse-free period, wherein said BurstDR electrical signal does not cause muscle back contraction; —optionally repeating the applying of the second electrical stimulation; thereby alleviating or eliminating the back pain.

According to an embodiment of this method, the consistent frequency of the first tonic signal is about 2 Hz to about 20 Hz, preferably of 2 Hz to about 10 Hz; or preferably of 2 Hz, 3 Hz, 4 Hz, 5 Hz, 6 Hz, 7 Hz, 8 Hz, 9 Hz, or 10 Hz.

According to another aspect, the invention also concerns a method for treating or alleviating back pain in a patient, the patient having epidurally implanted a rod-electrode, the method comprising the steps of:—applying, over a certain first time period, a first electrical stimulation via the rod-electrode, wherein said first electrical stimulation is a first tonic electrical signal having a consistent frequency of about 2 Hz to about 35 Hz, and, via the first tonic electrical signal, causing a contraction of the patient's both superficial and deeper lying back's muscle groups; —optionally repeating the applying of the first electrical stimulation; —applying, over a certain second time period, a second electrical stimulation via the rod-electrode, wherein said second electrical stimulation is a second tonic or high frequency electrical signal having a consistent frequency between about 40 Hz and 10000 Hz, wherein said second tonic or high frequency electrical signal does not cause muscle back contraction; —optionally repeating the applying of the second electrical stimulation; thereby further improving the alleviating or eliminating the back pain.

According to an embodiment of this method, the consistent frequency of the first tonic signal is about 2 Hz to about 20 Hz, preferably of 2 Hz to about 10 Hz; or preferably of 2 Hz, 3 Hz, 4 Hz, 5 Hz, 6 Hz, 7 Hz, 8 Hz, 9 Hz, or 10 Hz.

Also, the invention concerns a method for treating or alleviating back pain in a patient, the patient having epidurally implanted a plate electrode, the method comprising the steps of:—applying, over a certain first time period, a first electrical stimulation via the plate electrode, wherein said first electrical stimulation is a first tonic electrical signal having a consistent frequency of about 2 Hz to about 35 Hz, and, via the first tonic electrical signal, causing a contraction of the patient's both superficial and deeper lying back's muscle groups; —optionally repeating the applying of the first electrical stimulation; —applying, over a certain second time period, a second electrical stimulation via the plate electrode, wherein said second electrical stimulation is a second tonic or high frequency electrical signal having a consistent frequency of between about 40 Hz and 10000 Hz, wherein said tonic or high frequency electrical signal does not cause muscle back contraction; —optionally repeating the applying of the second electrical stimulation; thereby alleviating or eliminating the back pain.

According to an embodiment of this method, the consistent frequency of the first tonic signal is about 2 Hz to about 20 Hz, preferably of 2 Hz to about 10 Hz; or preferably of 2 Hz, 3 Hz, 4 Hz, 5 Hz, 6 Hz, 7 Hz, 8 Hz, 9 Hz, or 10 Hz.

### BRIEF DESCRIPTION OF THE DRAWINGS

FIG. 1 is a sonographic picture from a patient's back during application of MuscleSCS demonstrating the contraction of multiple layers of the patient's back muscles, i.e. superficial and deeper laying ones.

FIG. 2 depicts a diagram showing the pain intensity as a numerical rating score (NRS) from 0 to 10 in week 2 during the first study phase of the clinical study performed by the applicant for burst stimulation alone (B), MuscleSCS alone (M), burst stimulation combined with MuscleSCS (BM) and base line (X axis), plotted against the pain intensity NRS (y axis);

FIG. 3 depicts a diagram showing the pain disability index (PDI) at time points F0 (before implantation of the electrode), F3M (3 months of stimulation passed) and F6M (6 months of stimulation passed) (X axis), plotted against the total score PDI (Y axis).

FIG. 4 depicts a diagram showing the pain catastrophizing index (PCI) at time points F0 (before implantation of the electrode), F3M (3 months of stimulation passed) and F6M (6 months of stimulation passed) (X axis), plotted against the total score PCS (Y axis).

FIG. 5 shows a table showing the distribution of the percentages of the overall contentment after 6 months of the treated patients with the muscle stimulation.

### DESCRIPTION OF PREFERRED EMBODIMENTS

A magnitude of different types of pain can be treated by drugs, physiotherapy, surgery or broadly by pain therapy. Especially chronic pain is difficult to be treated. In case traditional therapies do not yield satisfying benefits, neuromodulation can be applied to alter the pain signalling to the central nervous system. For example, in spinal cord stimulation (SCS) an implanted electrode releases electrical signals to the spinal cord for the treatment of pain sensed at various body regions. However, most presently applied SCS methods are indicated to treat neuralgia (neuropathic pain) but not indicated to treat myalgia, e.g. chronic back pain that is caused by muscular tension. Said neuralgia is the perception of pain caused e.g., by unphysiological compression or stretching of nerves. Thus, neuralgia is caused by defects affecting the pain-signalling nerves as such However, other types of pain, especially myalgia, cannot be efficiently treated by the methods and devices of the prior art. Myalgia is caused by defects within the muscle tissue, which may be caused e.g., by the overuse of a certain muscle group. Within the medical context of the present invention i.e., chronic back pain, myalgia may occur as a consequence of muscle tension or long-term malposition of the patient's body.

The disclosed invention, which is referred to herein also as “MuscleSCS”, is a method for treating chronic pain in patients, preferably chronic back pain. The patient has implanted epidurally in the spinal canal an electrode, which may be part of a standard SCS device. The electrode can be implanted in the patient's spinal canal at a level between C1 to T12 including, by means between the first cervical vertebra and the twelfth thoracic vertebra including.

Standard low frequency SCS methods comprise the delivery of an electrical signal at a frequency of about 40 Hz to 70 Hz to the patient's spinal cord. The electrical signal is delivered via an implanted electrode positioned, such, that the stimulated area is congruent with the painful body region. This way, paresthesia is induced in the respective area, so that the pain is alleviated or inhibited. However, instead of the pain, the patient perceives a tingling in the respective area and the stimulation program needs to be adjusted dependent on the activity of the patient.

The latter drawbacks can be overcome by “high-frequency” SCS methods, disclosed for example in US 2012/0016437 A1, which is incorporated herein by reference, comprising the delivery of an electrical signal at a frequency of more than 5 kHz to the patient's spinal cord. Said high-frequency stimulation does not induce paresthesia in the treated painful body region and once an individual program is setup, it can be applied independent of the activity of the patient.

The “burst stimulation” is the delivery of an electrical signal consisting of groups of electrical pulses, called bursts, with a wavelike variation of frequency, amplitude and/or pulse width, disclosed e.g., in US 2021/0260365 A1, which is incorporated herein by reference, which better resembles the natural electrical signals of the central nervous system. Said burst stimulation, more specifically the BurstDR method, allows treating chronic back pain to a certain degree.

In the method of the invention, the type of electrode is not particularly limited and may be one of plate electrode or a rod-electrode. Preferably, the rod-electrode comprises 4, 8, or 16 contacts. Preferably, the plate electrode is a lamitrode, i.e. a percutaneous plate electrode.

Via said electrode, a first electrical stimulation is delivered to the patient's spinal cord. The MuscleSCS induces the contraction of the patient's both superficial and deeper laying back muscles dependent on the position of the electrode within the spinal canal as well as dependent on the respective contacts of the electrode via which the electrical signal is delivered. This way, the therapy is adjustable to the individual needs of a patient and specifically the painful muscle groups can be selected for treatment.

Said electrical stimulation, in the method of the invention, is the delivery of a first tonic electrical signal at a consistent frequency of 2 Hz to 35 Hz including, in other words the frequency may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 Hz. The first tonic electrical signal may be further described by having a consistent amplitude of 0.25 mA to 30 mA including or having a consistent pulse width of 10 μs to 500 μs including. The first tonic electrical signal may be optionally repeated multiple times.

Said electrical stimulation, in the method of the invention, the first tonic electrical signal can be stopped at any time and the stimulation can be paused for 1-600 seconds and then be resumed again, producing periods of ON-stimulation and periods of OFF-stimulation.

In the method of the invention, the MuscleSCS can be applied or used in combination with one or more SCS methods of prior art applying electrical signals characterized by other parameters and not inducing muscle stimulation, said SCS method preferably being a burst stimulation or a high-frequency stimulation, more preferably a BurstDR stimulation.

Said BurstDR stimulation used in combination with the MuscleSCS, in the method of the invention, comprises burst electrical signals described by having a frequency of 40 Hz to 500 Hz including, an amplitude of 0.05 mA to 1 mA including or a total duration of a burst of up to 1000 μs. A combination of the disclosed method and methods of prior art achieves beneficial synergistic effects.

Said high-frequency stimulation used in combination with the MuscleSCS comprises electrical signals delivered at a frequency of about 1 kHz to 10 kHz.

The term “combination” of MuscleSCS with burst and/or high-frequency SCS, is to be understood such that the respective stimulation programs are applied to a patient in an alternating manner. The stimulation programs must not be applied simultaneously to a patient. However, the type or frequency of alternation or the duration of periods for treatment with any of the stimulation programs during this alternation is not particularly limited.

Usually, patients having SCS devices implanted have the option to adjust the therapy duration or other parameters manually according to their needs or the respective settings have been programmed once after implantation by a physician. The MuscleSCS can be applied according to the patient's desire from about 1 minute to about 120 minutes including, preferably from about 10 minutes to about 30 minutes.

When the MuscleSCS is used or applied in combination with one or more other SCS methods, preferably a burst stimulation, delivering at least a second electrical signal, the duration of said second electrical signal may be from about 10 minutes to about 22 hours, according to the individual needs of the patient.

The application or use of the MuscleSCS method as disclosed induces a muscle contraction, thereby generating a pleasant, massage-like perception for the patient. Most patients describe the MuscleSCS as being comparably pain-relieving as BurstDR stimulation, as will be described in more detail in the following examples. The application or use of muscle stimulation in combination with burst stimulation according to the invention, improves the analgesic effects of the treatment. In other words, the application of MuscleSCS together with other SCS methods yields a superior beneficial pain-relieving treatment as compared to the prior art.

### DETAILED DESCRIPTION OF THE FIGURES

### Example 1

An exploratory study was performed concerning the respective conditions to achieve a MuscleSCS, during which electrical signals of 2 Hz, 4 Hz, 6 Hz or 8 Hz frequency were delivered to the spinal cord of different patients via implanted SCS devices. The devices represented lamitrodes and octrodes, wherein an octrode is an electrode having 8 electrical contacts and a lamitrode is a small plate electrode also having 8 electrical contacts.

As measures for the resulting back's muscle contraction palpation, video recording, electromyography and sonography were used. Additionally, the perception by the patient was measured on a scale from 1 (very pleasant) to 4 (very unpleasant). In all patients, a back muscle contraction was achieved. Depending on the position of the electrode and the use of different contacts of the electrode, specific muscle groups were stimulated (not shown in figures). About 80% of the patients described the MuscleSCS as pleasant.

FIG. 1 shows a sonographic picture acquired during application of the electrical signal on a patient's back. The scaling indicates the depth of the detected signal in the patient's back, wherein “0” marks the skin. Multiple layers of contracted muscles were detectable in various depth in the patient's back.

Conclusively, the inventors could prove that by delivering MuscleSCS to a patient's spinal cord, it is possible to selectively stimulate desired superficial and deeper laying patient's back muscles, which is perceived as pleasant.

### Example 2

The applicant conducted a further prospective, multicentric, single blinded, randomized crossover study with 58 adults suffering from chronic back pain, who had implanted a SCS device (58 patients were included in the study, 49 could be used for the final analysis). Prior to the study, the patients answered a questionnaire regarding the effects of the pain on their health and quality of life.

In a first 6-week study phase, the patients received randomized stimulation by the stimulation programs BurstDR stimulation alone, MuscleSCS alone or MuscleSCS combined with BurstDR stimulation, said treatment comprising only one of these stimulation programs at a time. The three different stimulation programs were applied each during a 2-weeks stimulation-period.

The program for MuscleSCS was applied in total two times per day, once at 9 am in the morning, once at 8 pm in the evening, for 30 minutes each. Said program was characterized by tonic electrical signals with a pulse frequency of 2 Hz to 35 Hz, an amplitude of 0.25 mA to 30 mA and a pulse width of 10 μs to 500 μs.

The program for BurstDR stimulation was applied constantly and was only interrupted when combined with the MuscleSCS such that at 9 am in the morning and at 8 pm in the evening, for 30 minutes each, the BurstDR stimulation was set off while the MuscleSCS was applied. Said BurstDR stimulation was characterized by a frequency of 40 Hz to 500 Hz, an amplitude lower than the amplitude of the tonic electrical signal during MuscleSCS and a pulse-free period in between the bursts.

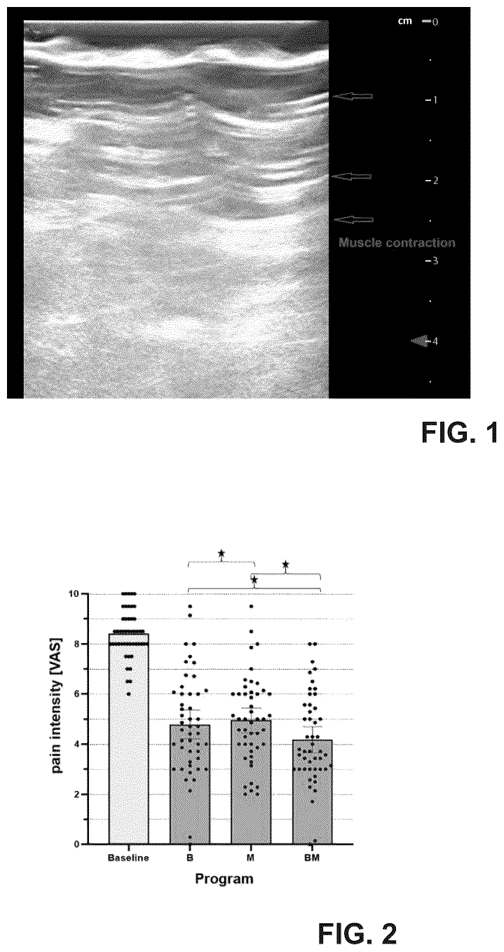

FIG. 2 shows the intensity of pain as judged by the patients (n=48) on a numerical scale from 0 (no pain) to 10 (maximum pain) after a one-week washout phase i.e., during the second week of each stimulation-block as well as the baseline pain intensity prior to treatment. The bars indicate the mean pain intensity and the whiskers indicate the 95% confidence interval. Treatment by each stimulation program achieves a statistically significant alleviation or inhibition of pain. The treatment by burst stimulation in combination with the MuscleSCS was most pain alleviating or inhibiting, thus most efficient.

In a second 6-week study phase the patients continued the treatment applying the stimulation program they preferred most (cross-over). At the end of this study phase, by means after a total duration of treatment of 3 months, the patients answered a second questionnaire regarding the effects of the pain and the treatment on their health.

In a third 3-month study phase, again, the patients had the option to switch from one stimulation program to another (cross-over) after having evaluated the foregone alleviation or inhibition of pain and continued the treatment with either the same stimulation program as in the second study phase or another desired stimulation program. At the end of this final study phase, by means after a total duration of treatment of 6 months, the patients answered a third questionnaire regarding the effects of the pain and the treatment on their health.

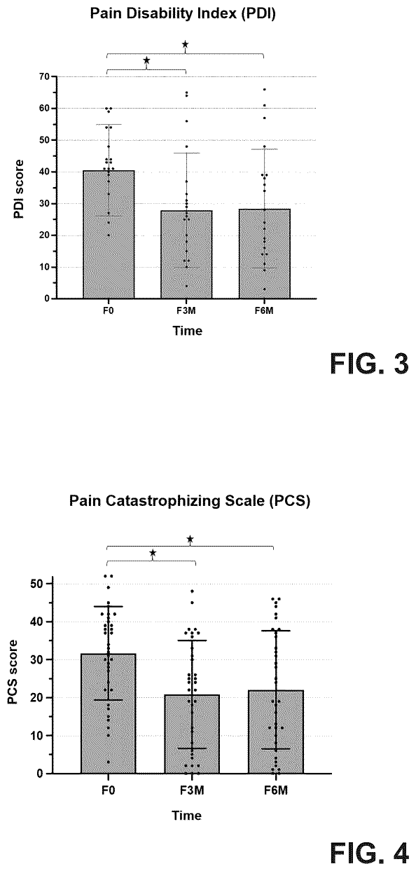

FIG. 3 shows data on the pain disability index (PDI) from the 3 mentioned questionnaires, the first (F0) answered prior to treatment, the second (F3M) answered after the second study phase, by means after 3 months of treatment, and the third (F6M) answered after the third study phase, by means after 6 months of treatment. The bars indicate the mean pain intensity and the whiskers indicate the 95% confidence interval. The PDI was significantly reduced after study phase 2 (F3M) and after study phase 3 (F6M) compared to the time prior to treatment (F0).

The PDI is a measure of the impact that pain has onto the participation of humans on the essential activities of life. By general definition, those activities comprise for example family- and household-activities, leisure activities, social interaction, social activities, work-related activities, sexual activities, personal care as well as the quality of basic activities as eating, sleeping, breathing. Those dimensions are rated on a numerical scale from 1 to 10 such that the PDI is the sum of the ratings of all dimensions. The higher the PDI value, the more the pain impacts the life of a patient.

FIG. 4 shows data on the pain catastrophizing score (PCS) from the 3 mentioned questionnaires, the first (F0) answered prior to treatment, the second (F3M) answered after the second study phase, by means after 3 months of treatment, and the third (F6M) answered after the third study phase, by means after 6 months of treatment. The bars indicate the mean pain intensity and the whiskers indicate the 95% confidence interval. The PCS was significantly reduced after study phase 2 (F3M) and after study phase 3 (F6M) compared to the time prior to treatment (F0).

The PCS is a measure of the catastrophizing of the pain by the patient. “Catastrophizing” is to be understood as the expectation of the incidence of a negative, by means painful, event. Multiple dimensions from three categories, by means magnification, rumination and helplessness are considered. Those dimensions are rated on a numerical scale from 0 to 4 such that the PCS is the sum of the ratings of all dimensions. The higher the PCS value, the more the patient catastrophizes the pain.

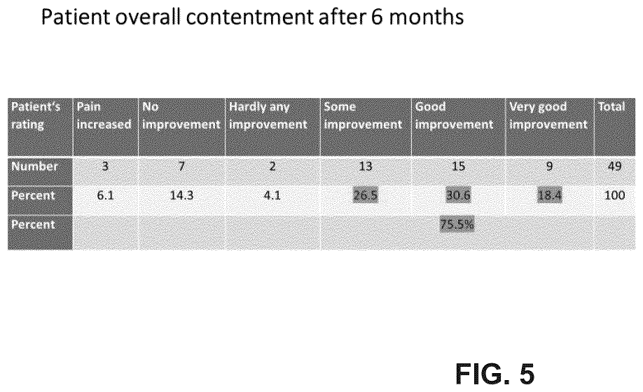

FIG. 5, in a table, shows the percentages of the contentment of the patients with the MuscleSCS on a scale from 0 (aggravates pain) to 5 (alleviates or inhibits pain very much) (in detail: 0=aggravates pain; 1=is not helpful; 2=scarcely alleviates or inhibits pain; 3=alleviates or inhibits pain to a certain degree; 4=alleviates or inhibits pain well; 5 alleviates or inhibits pain very much). As can be taken from table 1, about 75% of the patients experienced an alleviation or inhibition of their pain, demonstrating the advantageousness of MuscleSCS. About 71.4% of the patients preferred in study phases two and three a combined treatment by burst and MuscleSCS.

Conclusively, MuscleSCS alleviated or inhibited the chronic back pain of the patients. Especially, MuscleSCS combined with burst stimulation improved the beneficial outcome of the treatment for the patients with regard to alleviation or inhibition of chronic back pain.

From the foregoing, it will be appreciated that specific examples of the invention have been described herein for purposes of illustration, but that various modifications may be made without deviating from the spirit and scope of the invention. Accordingly, the invention is not limited except as by the appended claims.

Therefore, what is claimed is:

1. Method for treating or alleviating back pain in a patient, the patient having epidurally implanted an electrode, the method comprising the steps of:
applying, over a certain first time period, a first electrical stimulation via an electrode, wherein said first electrical stimulation is a first tonic electrical signal having a consistent frequency of about 2 Hz to about 35 Hz, and, via the first tonic electrical signal, causing a contraction of the patient's both superficial and deeper lying back's muscle groups;
optionally repeating the applying of the first electrical stimulation;
thereby alleviating or eliminating the back pain.

2. The method of claim 1, wherein the first tonic electrical signal is applied at a consistent amplitude of about 0.25 mA to about 30 mA.

3. The method of claim 1, wherein the first tonic electrical signal has a consistent frequency of about 2 Hz to about 35 Hz.

4. The method of claim 1, wherein the first tonic electrical signal has a consistent pulse width of about 10 μs to about 500 μs.

5. The method of claim 1, wherein the first tonic electrical signal, after a certain first time period, is paused for 1-600 seconds, and subsequently repeated again, thereby producing periods of ON-stimulation and periods of OFF-stimulation.

6. The method of claim 1, wherein the method further comprises a step of
applying, over a certain second time period, a second electrical stimulation via the electrode, wherein said second electrical stimulation is a BurstDR electrical signal, delivering groups of pulses at a frequency of 40 Hz and intraBurstDR 500 Hz and at amplitudes lower than the tonic electrical signal, wherein said groups of pulses are separated by a pulse-free period, wherein said BurstDR electrical signal does not cause muscle back contraction;
optionally repeating the applying of the second electrical stimulation;
thereby further improving the alleviating or eliminating the back pain.

7. The method of claim 6, wherein the second electrical stimulation is applied before or after a first electrical stimulation.

8. The method of claim 6 wherein the second electrical stimulation is repeatedly applied before or after repeatedly applying the first electrical stimulation.

9. The method of claim 6, wherein the second electrical stimulation is applied in alternating repeats with the first electrical stimulation.

10. The method of claim 6, wherein the BurstDR electrical signal, delivering groups of pulses at a frequency of 40 Hz and intraBurstDR of 500 Hz and at amplitudes between about 0.05 mA and 1 mA, and a duration of 1000 μs per impulse.

11. The method of claim 1, wherein the method further comprises a step of
applying, over a certain second time period, a second electrical stimulation via the electrode, wherein said second electrical stimulation is a second tonic or high frequency electrical signal having a consistent frequency between about 40 Hz and 10000 Hz, wherein said second tonic electrical signal does not cause muscle back contraction;
optionally repeating the applying of the second electrical stimulation;
thereby further improving the alleviating or eliminating the back pain.

12. The method of claim 1, wherein the certain first time period is from about 1 minute to about 120 minutes, preferably from about 10 minutes to about 30 minutes.

13. The method of claim 6, wherein the certain second time period is from about 10 minutes to about 22 h.

14. The method of claim 1, wherein the electrode is implanted at a patient's vertebral level between C1 to T12, preferably between T7 to T9.

15. The method of claim 1, wherein the electrodes are selected from a plate electrode and a rod-electrode, preferably a lamitrode.

16. A method for treating or alleviating back pain in a patient, the patient having epidurally implanted a plate electrode, the method comprising the steps of:
applying, over a certain first time period, a first electrical stimulation via the plate electrode, wherein said first electrical stimulation is a first tonic electrical signal having a consistent frequency of about 2 Hz to about 35 Hz, and, via the first tonic electrical signal, causing a contraction of the patient's both superficial and deeper lying back's muscle groups,
optionally repeating the applying of the first electrical stimulation,
applying, over a certain second time period, a second electrical stimulation via the plate electrode, wherein said second electrical stimulation is a BurstDR electrical signal, delivering groups of pulses at a frequency between about 40 Hz and intraBurstDR of 500 Hz, and at amplitudes lower than the tonic electrical signal, wherein said groups of pulses are separated by a pulse-free period, wherein said BurstDR electrical signal does not cause muscle back contraction,
optionally repeating the applying of the second electrical stimulation,
thereby alleviating or eliminating the back pain.

17. A method treating or alleviating back pain in a patient, the patient having epidurally implanted a rod-electrode, the method comprising the steps of:
applying, over a certain first time period, a first electrical stimulation via the rod-electrode, wherein said first electrical stimulation is a first tonic electrical signal having a consistent frequency of about 2 Hz to about 35 Hz, and, via the first tonic electrical signal, causing a contraction of the patient's both superficial and deeper lying back's muscle groups,
optionally repeating the applying of the first electrical stimulation,
applying, over a certain second time period, a second electrical stimulation via the rod-electrode, wherein said second electrical stimulation is a BurstDR electrical signal, delivering groups of pulses at a frequency of between about 40 Hz and intraBurstDR of 500 Hz and at amplitudes lower than the tonic electrical signal, wherein said groups of pulses are separated by a pulse-free period, wherein said BurstDR electrical signal does not cause muscle back contraction,
optionally repeating the applying of the second electrical stimulation,
thereby alleviating or eliminating the back pain.

18. A method for treating or alleviating back pain in a patient, the patient having epidurally implanted a rod-electrode, the method comprising the steps of:
applying, over a certain first time period, a first electrical stimulation via the rod-electrode, wherein said first electrical stimulation is a first tonic electrical signal having a consistent frequency of about 2 Hz to about 35 Hz, and, via the first tonic electrical signal, causing a contraction of the patient's both superficial and deeper lying back's muscle groups,
optionally repeating the applying of the first electrical stimulation,
applying, over a certain second time period, a second electrical stimulation via the rod-electrode, wherein said second electrical stimulation is a second tonic or high frequency electrical signal having a consistent frequency between about 40 Hz and 10000 Hz, wherein said second tonic or high frequency electrical signal does not cause muscle back contraction,
optionally repeating the applying of the second electrical stimulation,
thereby further improving the alleviating or eliminating the back pain.

19. A method for treating or alleviating back pain in a patient, the patient having epidurally implanted a plate electrode, the method comprising the steps of:
applying, over a certain first time period, a first electrical stimulation via the plate electrode, wherein said first electrical stimulation is a first tonic electrical signal having a consistent frequency of about 2 Hz to about 35 Hz, and, via the first tonic electrical signal, causing a contraction of the patient's both superficial and deeper lying back's muscle groups;
optionally repeating the applying of the first electrical stimulation;
applying, over a certain second time period, a second electrical stimulation via the plate electrode, wherein said second electrical stimulation is a second tonic or high frequency electrical signal having a consistent frequency of between about 40 Hz and 10000 Hz, wherein said tonic or high frequency electrical signal does not cause muscle back contraction;
optionally repeating the applying of the second electrical stimulation;
thereby alleviating or eliminating the back pain.
